(19) United States
(12) Patent Application Publication
(10) Pub. No.: US 2011/0318824 A1
(43) Pub. Date: Dec. 29, 2011

(54) CARTRIDGE SYSTEM FOR CHEMICAL PROCESSING

(75) Inventors: Takeo Tanaami, Tokyo (JP); Hisao Katakura, Tokyo (JP); Nobuyuki Kakuryu, Tokyo (JP)

(73) Assignee: YOKOGAWA ELECTRIC CORPORATION, Tokyo (JP)

(21) Appl. No.: 13/149,195

(22) Filed: May 31, 2011

(30) Foreign Application Priority Data
May 31, 2010 (JP) 2010-124192
Jan. 31, 2011 (JP) 2011-018173

(51) Int. Cl. C12M 1/02

(52) U.S. Cl. 435/303.1; 435/306.1

(57) ABSTRACT

An end portion of a head has a recess having an inner shape corresponding to a dome shape of a well when the well is in an expanded state. The radius of curvature of the recess is larger than the radius of curvature of the dome shape of the well. Moreover, the depth of the recess is smaller than the height of the dome shape of the well. Therefore, the head first contacts the apex of the well, and the contact area gradually expands toward the outer periphery. At this time, a gap is generated between the head and the cartridge around the well. Thus, the air between the head and the well is expelled from around the well as the head is pressed against the well.

### FIELD OF THE INVENTION

The present invention relates to a cartridge system for chemical processing that moves a solution in a cartridge and performs chemical processing in a well (chamber) formed in the cartridge by using deformation of the cartridge when an external force is applied to the cartridge. In particular, the present invention relates to a cartridge system for chemical processing that heats or cools a solution in a specific well.

### DESCRIPTION OF THE RELATED ART

A cartridge system for chemical processing has been developed in which a chemical process is performed by moving a content (solution) in a cartridge by using deformation of the cartridge when an external force is applied to the cartridge (see, for example, Japanese Unexamined Patent Application Publication No. 2005-024516). The cartridge system has wells and a channel connecting the wells formed therein. The cartridge system successively moves the solution from one well to another through the channel by using deformation of the cartridge when an external force is applied to the cartridge, thereby performing chemical processing. With the cartridge system, the structure of the cartridge itself serves to define the protocol of the chemical processing and keep a hermetic state, whereby a desired protocol can be performed safely without individual difference among operators.

### PRIOR ART REFERENCE

Japanese laid-open patent publication No. 2005-024516

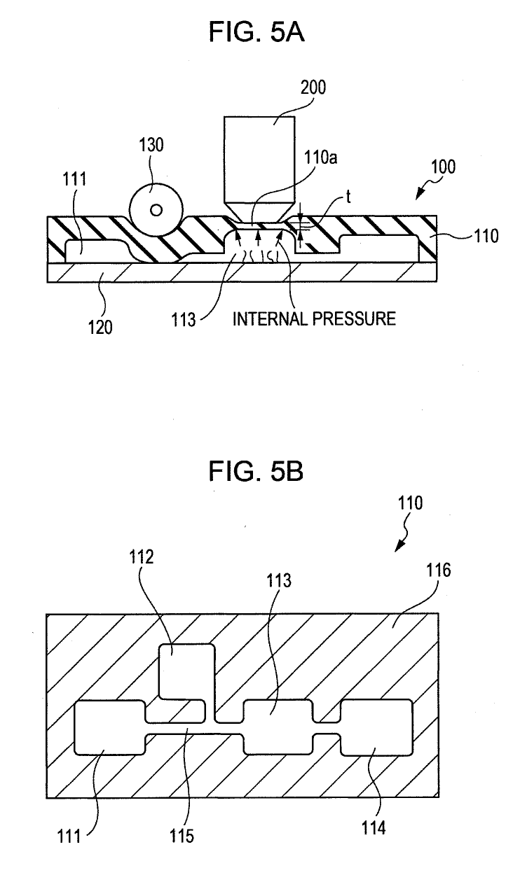

FIG. 5 includes schematic views of an example of an conventional cartridge system for chemical processing. The cartridge system has a structure that is suitable for amplifying genes in a cartridge by using a polymerase chain reaction (PCR) method and for handling a sample including DNA-bonded magnetic particles in the cartridge.

FIG. 5A is a longitudinal sectional view of the cartridge, and FIG. 5B is a plan view illustrating the disposition of chambers (wells), a channel, and the like.

A chemical reaction cartridge 100 includes an elastic body 110, which is made of a hermetic and elastic rubber, and a planar substrate 120, which is made of a hard material.

As illustrated in FIG. 5B, holes 111 and 112 for holding a solution (hereinafter referred to as chambers), a chamber 113 serving as a reaction section (hereinafter referred to as a reaction chamber), a chamber 114 for holding waste liquid (hereinafter referred to as a waste liquid chamber), and a channel 115 connected to the chambers are formed in the back surface of the elastic body 110 so as to be recessed toward the front surface of the elastic body 110.

A bonding region 116, which is a planar portion of the elastic body 110 other than the chambers and the channel, is bonded to a surface of the substrate 120. Thus, the elastic body 110 and the substrate 120 seal the chambers and the channel, thereby preventing leakage of the solution from the cartridge.

How a solution is moved in the cartridge is described. A roller 130 is pressed against a left-end part of the cartridge 100 to the extent that the chamber 111 becomes flattened. The roller 130 presses the cartridge 100 over the entire width of the cartridge 100. When the roller 130 is rotated and moved rightward from the left-end part of the cartridge 100 in this state, the solution stored in the chamber 111 and the solution stored in the chamber 112 are pushed rightward. These solutions are moved into the reaction chamber 113 through the channel 115.

An external jig 200 is a tool that heats, cools, or vibrates a sample (solution) in a chamber (for example, the reaction chamber 113 in FIG. 5A) by contacting an elastic film 110a, which is a thin part of the cartridge, at an end portion thereof. The sample is a solution including, for example, DNA and magnetic particles.

The elastic film 110a is provided only at a part of the cartridge above the chamber 113 that the external jig 200 contacts. The elastic film 110a is thinner than other parts of the elastic body, and has a thickness of 1 mm or less. The optimal film thickness t is, for example, in the range of 0.1 to 1 mm.

Next, the operation of the cartridge system is described. A sample (solution) is introduced into the chamber 113 of the cartridge, and the internal pressure of the chamber 113 is increased by blocking the channel and the like in the cartridge. At this time, the elastic film 110a is stretched.

The internal pressure can be increased not only in the chamber 113 alone but also in all of the chambers and the channel in the cartridge.

The end portion of the external jig 200 is pressed against and made to closely contact the surface of the elastic film 110a that is stretched by the internal pressure. The sample can be heated, cooled, or vibrated in accordance with the processing.

If the processing is to amplify genes by using a PCR method, heating and cooling of the sample are repeatedly performed. Because the heating and cooling is performed directly through the elastic film 110a, which is a thin film that closely contacts the external jig 200, the heating and cooling can be performed considerably faster than conventional indirect heating and cooling methods.

### SUMMARY OF THE INVENTION

### Objectives of the Invention

As described above, the temperature of a solution in the chamber 113 can be controlled by pressing the external jig 200 for heating and cooling from outside the chamber 113. The temperature of the external jig 200 can be controlled by using a Peltier element or the like.

However, as described above, the cartridge system in which a planar head (external jig) is pressed against the cartridge has a problem in that the cartridge system cannot control the temperature of the solution in the well rapidly and stably if a cartridge having a well that expands when the solution flows into the well is used, because, in such a case, a surface of the well that contacts the head is not flat and the well and the head do not sufficiently contact each other.

An object of the present invention is to provide a cartridge system for chemical processing that can control the temperature of a solution rapidly and stably.

According to the present invention, there is provided cartridge system for a chemical processing that moves a solution in a cartridge by using deformation of the cartridge when an external force is applied to the cartridge and performs chemical processing in a well formed in the cartridge. The cartridge system for chemical processing includes a cartridge including two substrates that are partially affixed to each other, at least one of the substrates being disposed on one side of the cartridge and made of an elastic material, the cartridge having a channel and the well that are formed therein between the two substrates, the channel allowing the solution to flow therethrough, the well holding the solution by expanding into a dome shape when the solution flows thereinto; and a head having a recess having an inner shape corresponding to the dome shape of the well when the well is in an expanded state, the head enclosing the well and heating or cooling the solution held in the well. With the cartridge system for chemical processing, the inner surface of the head closely contacts the outer periphery of the well because the inner surface of the head has a shape corresponding to the dome shape of the well when the well is in an expanded state, whereby the temperature of the solution in the well can be controlled rapidly and stably.

The shape of the recess in the head can have a radius of curvature that is larger than a radius of curvature of the dome shape of the well, and a depth of the recess can be smaller than a height of the dome shape of the well.

The well of the cartridge can expand into a dome shape toward each side of the substrates, and the head can include a pair of heads that face each other with the substrates therebetween and sandwich the well from both sides.

The head can be pressed against the well while enclosing the well, can pressurize the solution held in the well, and can heat or cool the solution.

The dome shape of the well can have a cavity in a central part thereof.

The dome shape of the well can include convex and concave portions.

In the cartridge system for chemical processing of the present invention, the inner surface of the head closely contacts the outer periphery of the well because the inner surface of the head has a shape corresponding to the dome shape of the well when the well is in an expanded state, whereby the temperature of the solution in the well can be controlled rapidly and stably.

### DESCRIPTION OF THE PREFERRED EMBODIMENTS

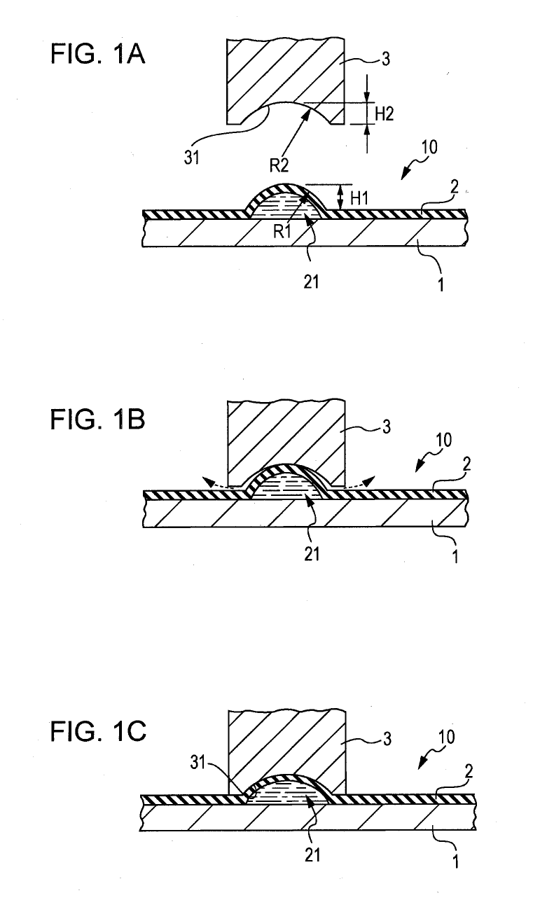

FIG. 1A illustrates a sectional view of a cartridge system for chemical processing according to an embodiment, FIG. 1B illustrates a state in which a head is pressed against a well, and FIG. 1C illustrates a state in which the head is completely pressed against the well.

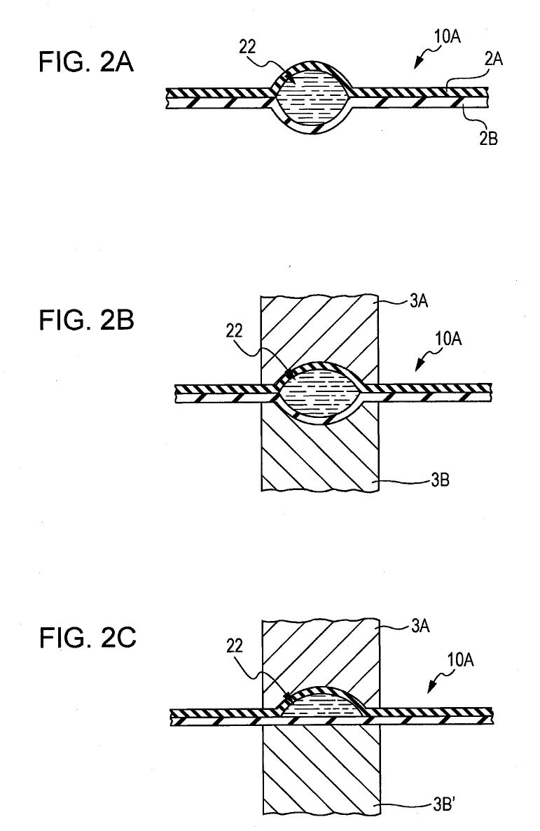

FIG. 2A is a sectional view illustrating an example in which a pair of heads facing each other are pressed against a well from both sides of the well, FIG. 2B illustrates a state in which a solution is heated or cooled, and FIG. 2C illustrates an example in which one of the heads has a flat surface.

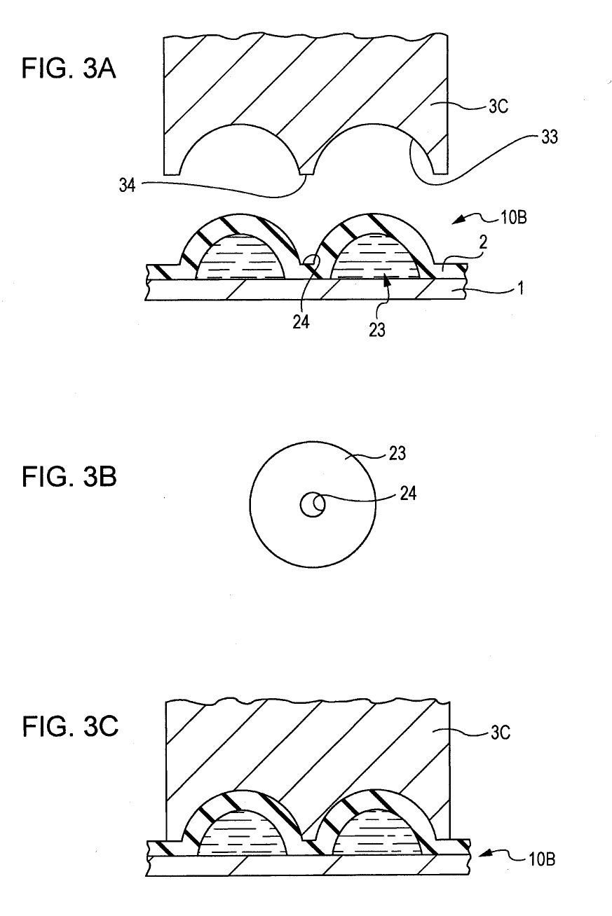

FIG. 3A is a sectional view illustrating an example of a doughnut-shaped well, FIG. 3B is a plan view of the doughnut-shaped well, and FIG. 3C illustrates a state in which a head is completely pressed against the well.

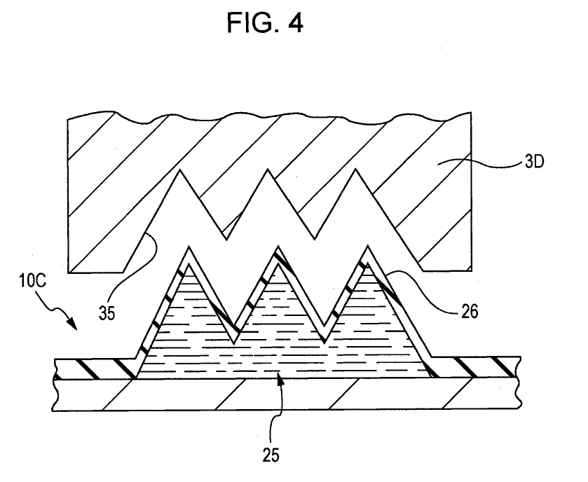

FIG. 4 is a sectional view illustrating an example of a well having convex and concave portions; and

FIGS. 5A and 5B are schematic views of an example of a conventional cartridge system for chemical processing.

### DESCRIPTION OF THE PREFERRED EMBODIMENTS

Hereinafter, an embodiment of the cartridge system for chemical processing of the present invention is described with reference to FIGS. 1A to 4.

FIG. 1A is a sectional view of a cartridge system for chemical processing according to the embodiment.

As illustrated in FIG. 1A, a chemical processing cartridge 10 according to the present embodiment includes a substrate 1 (first substrate) and an elastic member 2 (second substrate) superposed on the substrate 1.

The substrate 1 and the elastic member 2 are partially affixed to each other. A well 21 is a part of the elastic member 2 that is separated from the substrate 1 and expands into a dome shape toward a surface of the cartridge (upward in FIG. 1A) when a solution flows into the well 21. As illustrated in FIGS. 1A and 1B, the well 21 provides a space between the substrate 1 and the elastic member 2 and thereby holds the solution in the cartridge 10. A plurality of wells and a channel connecting the wells are formed in the cartridge 10 and provide spaces between the substrate 1 and the elastic member 2. The well 21 is one of these wells.

A method of using the cartridge 10 according to the present embodiment is described.

When a roller (not shown) or the like is pressed against the cartridge 10, the elastic member 2 is elastically deformed, and the space between the substrate 1 and the elastic member 2 becomes flattened. The flattened region moves as the roller moves, and the solution in the cartridge 10 is moved through the wells and the channel. After the solution has reached the well 21, the solution can be contained in the well 21 by pressing the roller to, for example, channel portions of the cartridge 10 before and behind the well 21.

As illustrated in FIG. 1A, an end portion of a head 3 has a recess 31 having a shape corresponding to that of the dome shape of the well 21 in an expanded state. The radius of curvature R2 of the recess 31 is larger than the radius of curvature R1 of the dome shape of the well 21. The depth H2 of the recess 31 is smaller than the height H1 of the dome shape of the well 21.

The temperature of the head 3 is controlled by using a Peltier device that is embedded in the head 3. By pressing the head 3 against the well 21 when the solution is contained in the well 21, the temperature of the solution in the well 21 can be controlled by heat conduction through the elastic member 2. The head 3 and a mechanism for controlling the temperature and position of the head 3 can be included in a driving device for moving the solution in the cartridge 10 as described above.

FIG. 1B illustrates a state in which the head 3 is pressed against the well 21. Because the inner shape of the recess 31 in the head 3 corresponds to the dome shape of the well 21 as described above, the head 3 first contacts the apex of the well 21 and then the contact area gradually expands toward the outer periphery as illustrated in FIG. 1B. At this time, a gap is generated between the head 3 and the cartridge 10 around the well 21. Thus, the air between the head 3 and the well 21 is expelled from around the well 21 as the head 3 is further pressed against the well 21.

FIG. 1C illustrates a state in which the head 3 is completely pressed against the well 21. In this state, the head 3 encloses the well 21, the entire surface of the recess 31 in the head 3 contacts the surface of the well 21, and the recess 31 is further pressed against the well 21. As a result, the well 21 is prevented from being expanded further, and even if a vapor or the like is generated in the well 21, expansion of bubbles in the well 21 is prevented.

In this state, the well 21 is sealed because the end portion of the head 3 is pressed against a region surrounding the well 21. Therefore, even when the solution in the well 21 is heated and the internal pressure is increased, the solution does not leak from the well 21 to the outside.

Thus, the entire surface of the recess 31 in the head 3 closely contacts the well 21, the contact area is increased, and thereby the heat conduction efficiency can be increased. Accordingly, the temperature of a fluid in the well 21 can be changed rapidly. When necessary, the head 3 can be completely pressed against the well 21 after repeating the operation of expelling air as illustrated in FIG. 1B. Moreover, the solution in the well 21 can be pressurized by pressing the head 3 against the well 21.

FIG. 2A is a sectional view illustrating an example in which a pair of heads facing each other are pressed against a well from both sides of the well. As illustrated in FIG. 2A, a cartridge 10A includes an elastic member 2A and an elastic member 2B. The elastic members 2A and 2B are partially bonded to each other and form a well 22 that expands into a dome shape toward each side of the substrates. As illustrated in FIG. 2A, the well 22 protrudes evenly toward both sides of the cartridge 10A when a solution flows into the well 22.

FIG. 2B illustrates a state in which the solution is heated or cooled. As illustrated in FIG. 2B, a pair of heads 3A and 3B, each having a Peltier device embedded therein, sandwich the well 22 from both sides of the cartridge 10A. Thus, the well 22 is enclosed by the heads 3A and 3B, and the solution in the well 22 is heated or cooled.

The relationship between the shapes (sizes) of recesses in the heads 3A and 3B, which enclose the well 22, and the shape (size) of the well 22 is similar to that in the case illustrated in FIG. 1A. Therefore, as the heads 3A and 3B are pressed against the well 22, the air between the head 3A and the well 22 and the air between the head 3B and the well 22 are expelled from around the well 22. When the heads 3A and 3B are completely pressed against the well 22, the recesses in the heads 3A and 3B closely contact the well 22. Therefore, further expansion of the well 22 is prevented, and even if a vapor or the like is generated in the well 22, expansion of bubbles in the well 22 is prevented.

In the examples illustrated in FIGS. 2B and 2C, the heads 3A and 3B are pressed against the well 22 from both sides of the well 22. Therefore, the solution in the well 22 can be heated or cooled more rapidly. In particular, this is effective for processing that requires a rapid change in the temperature, such as PCR, and a predetermined process can be performed in a short time. The solution in the well 22 can be pressurized by pressing the heads 3A and 3B against the well 22.

As illustrate in FIG. 2C, an end portion of one of the heads that sandwich the well 22 therebetween (here, a head 3B′) can have a flat surface.

FIG. 3A is a sectional view illustrating an example of a doughnut-shaped well 23, and FIG. 3B is a plan view of the doughnut-shaped well.

As illustrated in FIGS. 3A and 3B, a cavity 24 is formed in a central part of the well 23 in a cartridge 10B. As illustrated in FIG. 3B, the well 23 has a doughnut shape that is centered around the cavity 24 when viewed from above the cartridge 10B.

As illustrated in FIG. 3A, a recess 33 for enclosing the well 23 and a protrusion 34 corresponding to the cavity 24 are formed in a head 3C for heating or cooling the solution.

The relationship between the size of the recess 33 in the head 3C and the size of the well 23 is similar to that in the case illustrated in FIG. 1A. The radius of curvature of the recess 33 is larger than the radius of curvature of the dome shape of the well 23. The depth of the recess 33 is smaller than the height of the dome shape of the well 23. Therefore, the air between the head 3C and the well 23 is expelled from around the well 23 while the head 3C is being pressed against the well 23. As illustrated in FIG. 3C, the recess 33 in the head 3C closely contacts the well 23 when the head 3C is completely pressed against the well 23. Therefore, further expansion of the well 23 is prevented, and even if a vapor or the like is generated in the well 23, expansion of bubbles in the well 23 is prevented.

As illustrate in FIG. 3C, the protrusion 34 of the head 3C enters the cavity 24 in the well 23 when the head 3C is pressed against the well 23, whereby the inner surface of the head 3C closely contacts the well 23. Therefore, the contact area between the head 3C and the well 23 is increased, the heat conduction efficiency is improved, and the temperature of the solution in the well 23 can be changed more rapidly.

FIG. 4 is a sectional view illustrating an example of a well having convex and concave portions.

As illustrated in FIG. 4, an outer surface 26 of a well 25 in a cartridge 10C has convex and concave portions. An inner surface 35 of a head 3D, which is to be pressed against the outer surface 26 of the well 25, has a similar shape.

The head 3D and the well 25 are designed so that the inner surface 35 of the head 3D closely contacts the outer surface 26 of the well 25 when the head 3D is pressed against the well 25. In the example illustrated in FIG. 4, the contact area between the well 25 and the head 3D is increased due to the convex and concave portions, and the heat conduction efficiency can be increased. Therefore, the temperature of the solution in the well 25 can be changed further rapidly.

In the description above, the cartridge system for chemical processing according to the present invention is applied to PCR amplification that amplifies genes while changing the temperature of the well. However, the application of the present invention is not limited thereto, and the present invention can be used for nucleic acid extraction for eluting nucleic acid from a cell.

For example, a cell suspension is introduced into the well, the well is sealed by pressing the head, and the inside of the well is heated to a high temperature. The membrane structure of cells is weakened by heating the cell suspension to a high temperature, and elution of nucleic acid is accelerated.

The temperature and time for heating the well are determined at optimal values in accordance with the type of the cells. Also, the head can be provided with a cooling function, so that the temperature of the well can be rapidly decreased after being increased to a desired temperature.

The internal pressure of the well, which has been increased by heat, can be rapidly reduced by rapidly removing the pressing force of the head to release the sealed state of the well after increasing the temperature of the well. Elution of nucleic acid can be further accelerated by releasing the pressure after heating.

As described above, with the cartridge system for chemical processing according to the present invention, the inner surface of the head closely contacts the outer periphery of the well because the inner surface has a shape corresponding to the dome shape of the well when the well is in an expanded state, whereby the temperature of a solution in the well can be rapidly and stably controlled.

The application of the present invention is not limited to the embodiment described above. The present invention can be applied to a variety of cartridge system for chemical processings that moves a solution in a cartridge by using deformation of the cartridge when an external force is applied to the cartridge and that performs chemical processing in a well (chamber) formed in the cartridge.

1-6. (canceled)

7. A cartridge system for chemical processing, comprising:
a cartridge, comprising:
a first substrate made of a first elastic material;
a second substrate partially affixed to the first substrate, wells including a first well provided between the first substrate and the second substrate;
a channel provided between the first substrate and the second substrate, the channel connecting the wells to each other;
wherein when a solution is in the first well, the first substrate at the first well expands as a first dome; and
a first head comprising a first recess corresponding to the first dome to enclose the first well from an external side of the first substrate,
wherein the first recess heats or cools the solution inside the first well to perform a chemical process.

8. A cartridge system for chemical processing according to claim 7,
wherein the wells further includes a second well,
wherein the second well stores a solution,
wherein when the first substrate is elastically deformed at the second well, the solution as stored is flown into the first well.

9. A cartridge system for chemical processing according to claim 7,
wherein the first recess has a radius of curvature larger than a radius of curvature of the first dome,
wherein the first recess has a depth smaller than a height of the first dome.

10. A cartridge system for chemical processing according to claim 8,
wherein the second substrate is made of a second elastic member,
wherein when the solution is in the first well, the second substrate at the first well expands as a second dome, opposite to the first dome,
wherein the cartridge system further comprises a second head comprising a second recess corresponding to the second dome to enclose the first well from an external side of the second substrate.

11. A cartridge system for chemical processing according to claim 7,
wherein the first head is pressed against the first well to enclose the first well, the first head pressurizing the solution inside the first well.

12. A cartridge system for chemical processing according to claim 10, wherein the second head is pressed against the first well to enclose the first well, the second head pressurizing the solution inside the first well.

13. A cartridge system for chemical processing according to claim 7, wherein the first dome has a cavity at a central part thereof.

14. A cartridge system for chemical processing according to claim 7, wherein the first dome has a plurality of convex parts and concave parts.

15. A cartridge system for chemical processing according to claim 7, wherein when the first head is completely pressed against the first well, the first well is sealed.

16. A cartridge system for chemical processing according to claim 7, wherein when the first head is completely pressed against the first well, an entire surface of the first recess contacts the first well.
